(19) United States
(12) Patent Application Publication
(10) Pub. No.: US 2017/0007333 A1
(43) Pub. Date: Jan. 12, 2017

(54) System and Method for Monitoring the Movement of a Medical Instrument in the Body of a Subject

(71) Applicant: INRIA INSTITUT NATION DE RECHERCHE EN INFORMATIQUE ET EN AUTOMATIQUE, Le Chesnay (FR)

(72) Inventors: Mario Sanz Lopez, Lille (FR); Stéphane Cotin, Strasbourg (FR); Jérémie Dequidt, Lille (FR); Christian Duriez, Villeneuve d'Ascq (FR)

(21) Appl. No.: 15/109,034

(22) PCT Filed: Dec. 23, 2014
(86) PCT No.: PCT/FR2014/053540

§ 371 Date: Sep. 28, 2016

(30) Foreign Application Priority Data
Dec. 31, 2013 (FR) 1363755

(51) Int. Cl. A61B 34/20

(52) U.S. Cl. CPC A61B 34/20; A61B 2034/2055

(57) ABSTRACT

This system comprises means (9) for determining a position of a first portion of a medical instrument in the body of a subject at a determination time, and means (11) for displaying an image of the first portion in the determined position. The determination means (9) comprise an imaging module (15) that is capable of acquiring, at an acquisition time that precedes said determination time, a position of the first portion, a detection module (17) for detecting a movement of a second portion of the medical instrument between said acquisition and determination times, and a determination module (19) for determining the position of the first portion (3d) at said determination time, from said position of the first portion at said acquisition time and from said movement of the second portion.

The present invention relates to a system for tracking a first section of a medical instrument, which section is inserted into the body of a subject, as it is moved in the body of the subject, said system comprising means for determining a position of said first section with respect to the body of the subject at at least one determining time, and means for displaying, to a user, at said determining time, an image representative of at least one part of the body of the subject and of the first section of the medical instrument in the position of the first section determined by said determining means at the determining time.

It may in particular be used to guide the movement of medical instruments such as catheters, guides, needles or endoscopes in a blood vessel or a natural cavity of the body of a subject, during medical interventions. The success of these interventions especially depends on the precision of the movement of the medical instruments in the body of the subject.

Such an instrument is conventionally guided using scanner imaging techniques allowing the movement of the instrument in the body of the subject to be viewed.

These techniques are for example implemented by acquiring an initial image of the vascular system of the subject, before the intervention, and by superposing on this initial image successive images of the instrument as it is moved in the vascular system. These images are for example acquired at a frequency of 30 images per second.

The initial image of the vascular system is generally acquired by means of an angiographic scanner. To do this, a contrast agent that is opaque to x-rays, for example an iodinated product, must be injected beforehand into the vascular system of the subject. During the intervention, the successive images are also acquired by scanner, the instrument being opaque to x-rays.

Such techniques require x-rays to be repeatedly emitted toward the body of the subject and therefore present a risk to his health.

To minimize this risk, it is possible to decrease the frequency at which the images are acquired as the instrument is moved, for example to 15 or even 7.5 images per second. However, this solution leads to a degradation in the quality of the images provided to the practitioner, and especially to images that flicker and to a degradation in the precision of the displayed movement of the instrument.

To overcome these drawbacks, it is known to replace images obtained by scanner by images obtained by magnetic resonance imaging (MRI). However, this solution proves to be very costly.

Therefore, the aim of the invention is to overcome the aforementioned drawbacks and in particular to provide a system which allows the movement of a medical instrument in the body of a subject to be tracked with a high precision, which minimizes the risks run by the subject, and which is of low cost.

To this end, one subject of the invention is a system of the aforementioned type, characterized in that said determining means comprise:
an imaging module able to acquire, at at least one acquiring time prior to said determining time, a position of the first section of the medical instrument with respect to the body of the subject;
a module for detecting a movement of a second section of the medical instrument with respect to the body of the subject between said acquiring time and said determining time; and
a determining module able to determine, from the position of the first section at said acquiring time, which position is output by said imaging module, and from said movement of the second section of the medical instrument between said acquiring time and said determining time, which movement is detected by said detecting module, the position of the first section of the medical instrument with respect to the body of the subject at said determining time.

According to other aspects of the invention, the method has one or more of the following features:
said detecting module is able to detect a translation of said second section of the medical instrument in its longitudinal direction and a rotation of said second section of the medical instrument about its longitudinal direction with respect to the body of the subject between said acquiring time and said determining time;
said determining module is able to determine the position of the first section of the medical instrument with respect to the body of the subject, at each of a plurality of successive determining times comprised between first and second successive acquiring times, from the position of the first section at said first acquiring time, which position is output by said imaging module, and from the movement of the second section of the medical instrument between said first acquiring time and each determining time of said plurality of determining times, which movement is detected by said detecting module;
said detecting module comprises at least one detector of a movement of the second section with respect to this detector;
said detector is contained in a housing including a duct for passing the medical instrument;
said housing includes a first section enclosing said detector and a second section enclosing said passing duct;
said second section is leaktight, said medical instrument passing through said passing duct being sealably isolated from said first section;
said second section is removably mounted on said first section;
said detector is an optical detector;
said optical detector comprises at least one light source able to emit an incident light beam onto a region of the second section of the medical instrument and one optical receiver able to detect a light beam reflected by the second section of the medical instrument;
said light source is able to emit the incident light beam onto a region of the second section of the medical instrument during the passage of said second section through said passing duct;
said detector is movable with respect to the body of the subject, and said detecting module comprises means for detecting a movement of the detector with respect to the body of the subject;
said first section of the medical instrument comprises at least one region visible by optical imaging, and said imaging module comprises an emitter able to emit optical rays toward the body of the subject, and a detector able to receive the optical rays emitted by said emitter through the body of the subject;
said second section of said medical instrument is outside the body of the subject.

Another subject of the invention is a method for tracking a first section of a medical instrument inserted into the body of a subject as it is moved in the body of the subject, comprising:
determining a position of said first section with respect to the body of the subject at at least one determining time; and
displaying, to a user, at each determining time, an image representative of at least one part of the body of the subject and of the first section of the medical instrument in the position of the first section determined by said determining means at the determining time,
   the method being characterized in that the determination of the position of said first section comprises:
acquiring, at at least one acquiring time prior to said determining time, a position of the first section of the medical instrument with respect to the body of the subject;
detecting a movement of a second section of the medical instrument with respect to the body of the subject between said acquiring time and said determining time; and
determining, from the position of the first section at said acquiring time and from said movement of the second section of the medical instrument between said acquiring time and said determining time, the position of the first section of the medical instrument with respect to the body of the subject at said determining time.

The invention will be better understood on reading the following description, which is given merely by way of example and with reference to the appended drawings, in which:

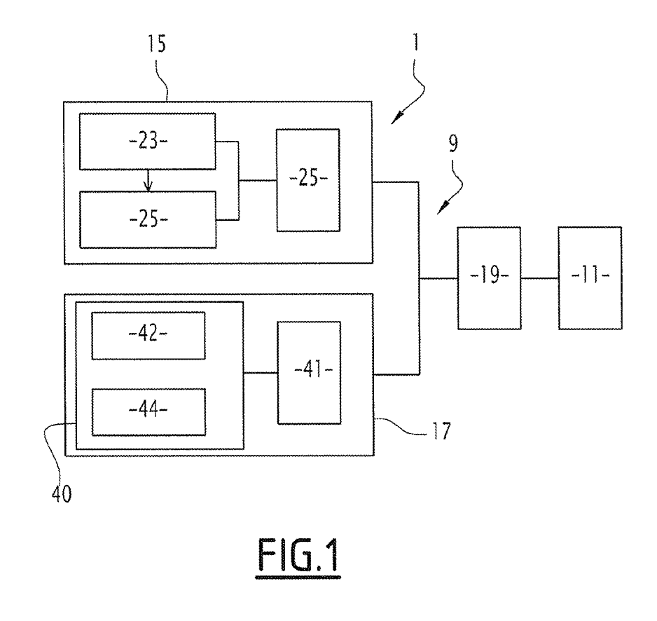

FIG. 1 is a block diagram of a tracking system according to one embodiment of the invention;

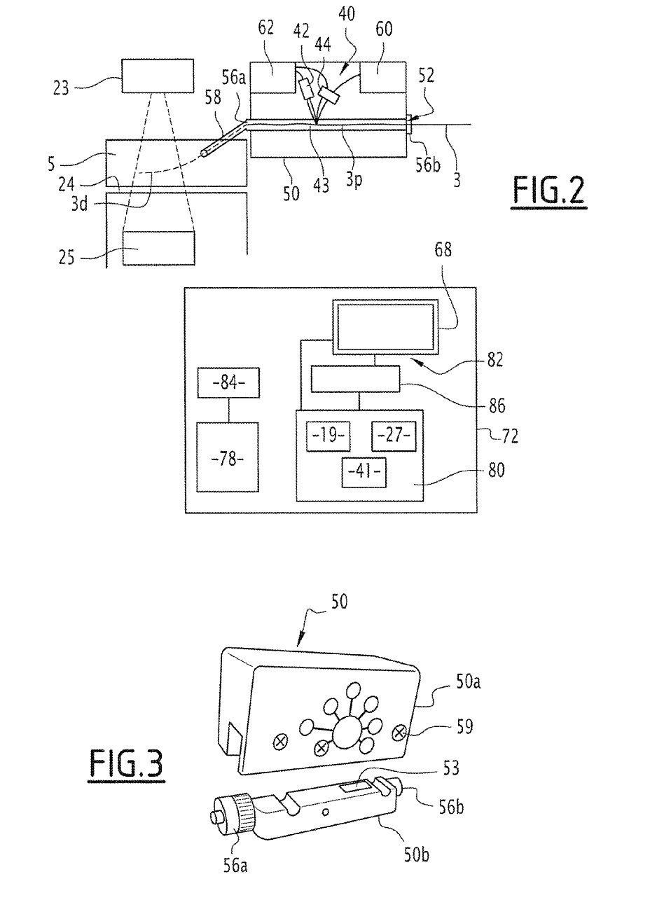

FIG. 2 is a diagram illustrating an exemplary implementation of the tracking system in FIG. 1;

FIG. 3 is a schematic perspective of a portion of the system in FIG. 2;

FIG. 4 is an exemplary image delivered by the system according to the invention; and

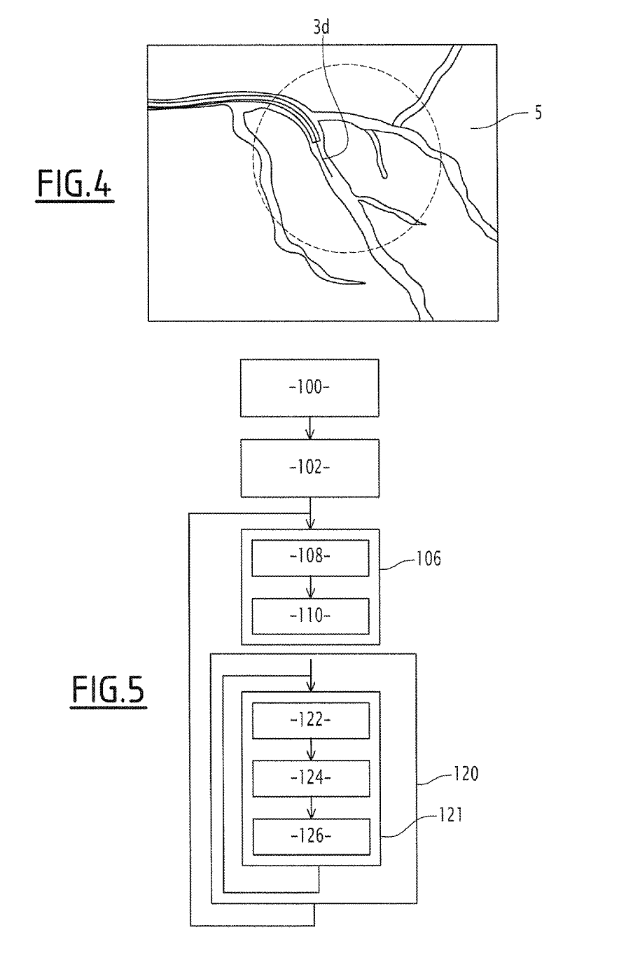

FIG. 5 is a block diagram of a tracking method implemented by the system in FIG. 1.

FIGS. 1 to 3 schematically shows a system 1 for tracking the movement of a medical instrument 3 in the body of a subject 5 according to one embodiment of the invention.

The medical instrument 3 is a flexible instrument of generally tubular shape, such as a catheter, a micro-catheter or a guide.

The medical instrument 3 is a flexible tube of substantially circular cross section, extending in a possibly curved longitudinal direction.

The medical instrument 3 is torsionally stiff about its longitudinal direction. Thus, rotating a section of this medical instrument 3 about its longitudinal direction causes the entirety of this medical instrument 3 to rotate about its longitudinal direction.

Moreover, translating a portion of the instrument 3 along its longitudinal direction causes the entirety of the instrument 3 to move.

In this embodiment, the medical instrument 3 in question is a catheter, and the system 1 according to the invention is used to track the movement of a portion of this catheter 3 in the vascular system of the subject 5.

The length of the catheter 3 is for example comprised between a few tens of centimeters and 2 meters, and its diameter is comprised between a few tenths of a millimeter and a few millimeters and especially between 0.5 mm and 5 mm.

In the rest of the description, the expression “distal section” 3d of the catheter 3 will be used to designate the portion of the catheter 3 introduced into and moved in the body of the subject 5, and the expression “proximal section” 3p of the catheter 3 will be understood to mean the section of the catheter remaining outside the body of the subject 5, this section being manipulated by an operator to move the distal section 3d in the body of the subject 5.

The catheter 3 is, in the present case, made from a material that is opaque to x-rays, for example a plastic such as a fluoropolymer.

The distal section 3d of the catheter 3 is for example introduced into an artery or vein of the subject 5 through a trocar 58 attached to the skin of the subject 5.

The system 1 comprises means 9 for determining the position of the distal section 3d of the catheter with respect to the vascular system of the subject 5 at a plurality of determining times t_{d}, and means 11 for displaying the movement of the catheter 3 in the vascular system of the subject 5.

Preferably, the determining times t_{d} are regularly spaced, the position of the distal section 3d of the catheter 3 being determined by the means 9 and displayed by the display means 11 at a determining frequency f_{d} for example comprised between 20 and 40 images per second and especially equal to 30 images per second. Two successive determining times will be denoted t_{d}(k-1) and t_{d}(k) below.

The means 9 include an imaging module 15 able to acquire, at a plurality of successive acquiring times tₐ, the position of the distal section 3d of the catheter 3 as it is moved in the vascular system of the subject 5.

The acquiring times tₐ are times such that at least one determining time t_{d} is comprised between two acquiring times tₐ.

Preferably, the acquiring times tₐ are regularly spaced, the position of the distal section 3d of the catheter 3 being acquired by the imaging module 15 at an acquiring frequency fₐ that is lower than the determining frequency f_{d}. The acquiring frequency fₐ is for example comprised between 2 and 10 images per second. The acquiring frequency fₐ is for example a sub-multiple of the determining frequency f_{d}.

Two successive acquiring times will be denoted tₐ(n−1) and tₐ(n) below.

The means 9 furthermore include a module 17 for detecting the movement of the proximal section 3p of the catheter 3 between two successive determining times t_{d}, and a module 19 for determining the position of the distal section 3d of the catheter 3, at each determining time t_{d}, from the positions of this distal section 3d at each acquiring time tₐ, which positions are acquired by the imaging module 15, and from the movements of the proximal section 3p, which movements are output by the detecting module 17.

Thus, the determining times t_{d} at which the position of the distal section 3d of the catheter is determined comprise, apart from the acquiring times tₐ at which an image of this distal section 3d is acquired, intermediate times comprised between two successive acquiring times tₐ, the position of the distal section 3d of the catheter 3 at each intermediate time being determined from the movement of the proximal section 3p of the catheter 3.

The imaging module 15 for example comprises an x-ray imaging system comprising an x-ray emitter 23, an x-ray detector 25 and a processing and controlling unit 27 connected to the emitter 23 and to the detector 25.

The x-ray emitter 23 is for example an x-ray tube. The emitter 23 is positioned facing a table 24 for supporting the subject 5. It is able to emit, at each acquiring time tₐ, x-rays in the direction of the subject 5 stretched out on the supporting table, and in particular in the direction of the region of interest of the body of the subject 5, i.e. the region of his vascular system in which it is intended to move the catheter 3.

The x-ray detector 25 is placed facing the emitter 23, the supporting table being placed between the emitter 23 and the detector 25.

Thus, the x-ray detector 25 is able to receive x-rays emitted by the emitter 23 through the body of the subject 5. The catheter 3 is at least partially opaque to the x-rays.

Thus, when it is introduced into the vascular system of the subject 5, the x-rays that the catheter 3 receives from the emitter 23 are not transmitted to the detector 25. The detector 25 is able to send signals representative of the detected x-rays to the processing and controlling unit 27.

The unit 27 is able to command the emission of x-rays by the emitter 23 at each acquiring time tₐ, and to receive the signals output by the detector 25, which signals are representative of the x-rays detected by this detector 25 at this acquiring time tₐ, and to generate, from these signals, an x-ray image of the body of the subject 5. When the catheter 3 is present in the vascular system of the subject 5, the catheter 3, and in particular its distal section 3d, appears in the image generated by the processing and controlling device 27.

The vascular system of the subject 5 does not appear in this image because the latter is not opaque to x-rays.

The processing and controlling unit 27 is able to reconstruct an image of the vascular system of the subject 5 in which both the vascular system and the catheter 3 appear by superposing, on each x-ray image, an initial image of the vascular system of the subject 5. This initial image is for example an image acquired beforehand by the imaging module 15 after introduction of a contrast agent that is opaque to x-rays into the vascular system of the subject 5.

The processing and controlling unit 27 is furthermore able to determine, from this reconstructed image, the position of the catheter 3, and in particular of its distal section 3d, at the acquiring time tₐ, in a frame of reference R associated with the vascular system of the subject 5.

The detecting module 17 is able to detect any movement of the proximal section 3p of the catheter 3 with respect to the subject 5, and in particular with respect to the vascular system of the subject 5, between two successive determining times t_{d}.

To this end, the detecting module 17 comprises a movement detector 40 able to detect, between two successive determining times t_{d}, the relative movement of the proximal section 3p of the catheter 3 with respect to this detector 40 in two degrees of freedom corresponding, on the one hand, to a translation of the catheter 3 in its longitudinal direction, and on the other hand, to a rotation of the catheter about its longitudinal direction.

The detecting module 17 moreover comprises a unit 41 for processing data output by the detector 40 in order to deduce therefrom the movement of the proximal section 3p of the catheter 3 with respect to the frame of reference R associated with the vascular system of the subject 5, between two successive determining times t_{d}.

Preferably, and as illustrated in FIG. 2, the detector 40 is an optical detector. It comprises a laser emitter 42 able to emit a laser beam toward a predetermined detecting region 43, and an optical receiver 44 able to receive and to detect laser radiation output by the laser emitter 42 after reflection from the catheter 3.

The detecting region 43 is placed on the path along which the proximal section 3p of the catheter 3 passes as it is moved by an operator.

The laser emitter 42 for example comprises a laser diode able to emit a laser beam, through a lens, toward the detecting region 43. The laser beam emitted by the laser emitter 42 is therefore received and reflected by the exterior wall of the catheter 3.

The distance between the laser emitter 42 and the exterior wall of the catheter 3 is a fixed distance, for example comprised between 2.2 and 2.4 mm.

Preferably, the laser diode 48 emits in the infrared.

The optical receiver 44 comprises a matrix-array of sensors, for example CMOS or CCD sensors. The optical receiver 44 is for example formed open area of 32×32 sensors. The sensors are able, after reflection from the catheter 3, to receive the laser radiation output by the laser emitter 42 and to convert this radiation into electrical signals representative of the received light intensity.

The optical receiver 44 is thus able to acquire, at receiving times tᵣ, images of the section of the catheter 3 passing through the region 43, at a receiving frequency fᵣ higher than the determining frequency f_{d}. The receiving frequency fᵣ is for example comprised between 125 and 1000 images per second.

As illustrated in FIG. 2, the detector 40 is contained in a housing 50 enclosing the laser emitter 42 and the optical receiver 44, and comprising a duct 52 allowing the catheter 3 to pass, the detecting region 43 being placed in this duct 52.

Thus, a movement applied to the proximal section 3p of the catheter 3 by an operator, to move the distal section 3d of the catheter 3 in the body of the subject 5, induces a movement of the proximal section 3p through the duct 52, and in particular in the detecting region 43, thereby allowing the detector 40 to sense any movement of this proximal section 3p.

For example, as illustrated in FIG. 3, the housing 50 includes a first section 50a enclosing the laser emitter 42 and the optical receiver 44, which section is referred to as the sensor 50a below, and a second section 50b enclosing the duct 52, which section is removably mounted on the first section and referred to as the holder 50b below.

Preferably, the holder 50b is leaktight, such that the medical instruments passing through the duct 52 are sealably isolated from the sensor 50a and in particular the laser emitter 42 and the optical receiver 44.

The holder 50b is able to be sterilized in an autoclave. The duct 52 comprises an aperture allowing the laser beam output by the laser emitter 42 to pass toward the catheter 3. This aperture is for example formed by a transparent window 53 formed in one surface of the holder 50b.

Removably mounting the holder 50b of the housing 50 on the sensor 50a makes it possible to adapt, depending on the type and size of the medical instrument to be placed in the duct 52, various holders 50b to a given sensor 50a, and therefore to ensure the adaptability of the housing 50 to various medical instrument.

In particular, the dimensions of the holder 50b, which allow the position of the duct 52 with respect to the sensor 50a and the diameter of the duct 52 to be adjusted, are chosen depending on the diameter of the catheter 3 so as to guarantee an optimal distance between the laser emitter 42 and the exterior wall of the catheter 3.

Thus, the inside diameter of the duct 52 is chosen, depending on the outside diameter of the catheter 3, so as to guarantee the desired distance between the laser emitter 42 and the exterior wall of the catheter 3, which distance is for example comprised between 2.2 and 2.4 mm.

The holder 50b moreover comprises a first exterior connector 56a allowing the housing 50 to be attached to the medical instrument through which the catheter 3 is introduced into the vascular system, in the present case a trocar 58, and a second exterior connector 56b allowing the housing 50 to be attached to a hemostasis valve or another device that in conventional use would have been attached to the trocar 58.

The sensor 50a and the holder 50b are attached to each other by fastening means, screws 59 for example.

The housing 50 furthermore comprises a communication interface 60 allowing data captured by the detector 40 to be transferred to the processing unit 41. Preferably, this interface 60 is a wireless interface and for example a radiofrequency emitter.

Preferably, the detector 40 is powered by a battery 62 included in the housing. Thus, the housing 50 may be used without being connected by a wired connection to a power source or to the processing unit 41.

The housing 50 is preferably made from sintered polyamide, allowing it to be autoclaved.

The processing unit 41 is able to receive from the receiver 44 signals representative of the images acquired by this receiver 44, and to analyze these images in order to determine the relative movement of the proximal section 3p of the catheter 3, in a frame of reference R′ associated with the detector 40, between two successive determining times t_{d}.

In a known way, this analysis is carried out by determining a correlation between two images taken in succession by the receiver 44. This correlation allows the relative movement of the proximal section 3p of the catheter 3 with respect to the detector 40 in the aforementioned two degrees of freedom between two receiving times tᵣ to be detected.

The processing unit 41 is able to deduce the relative movement of the proximal section 3p of the catheter 3 in the frame of reference R′ associated with the detector 40 between two successive determining times t_{d} by composing the movements detected between the receiving times tᵣ comprised between these two successive determining times t_{d}.

Moreover, the processing unit 41 is able to determine the relative movement of the proximal section 3p of the catheter 3 in the frame of reference R of the vascular system of the subject 5 from the relative movement of this proximal section 3p in the frame of reference R′ of the detector 40.

In the embodiment shown in FIGS. 2 and 3, the housing 50 is attached to the trocar 58, which itself is attached to the skin of the subject 5. The housing 50 and the detector 40 therefore occupy a fixed position with respect to the vascular system of the subject 5. Therefore, the relative movement of the proximal section 3p of the catheter 3 in the frame of reference R of the vascular system of the subject 5 is identical to the relative movement of this proximal section 3p in the frame of reference R′ of the detector 40.

The optical receiver 44 for example has a resolution of 1200 dots per inch, i.e. 48 dots per millimeter, thereby allowing the translational and rotational movements of the proximal section 3p of the catheter 3 to be sensed with precision.

For example, the maximum detectable speed of movement is comprised between 100 and 1000 mm/s and especially equal to 378 mm/s.

The module 19 for determining the position of the distal section 3d is connected to the imaging module 15 and to the detecting module 17.

The module 19 is able to determine the position of the distal section 3d of the catheter 3 at each determining time t_{d}, from the positions of this distal section 3d, which positions are acquired by the imaging module 15, at each acquiring time tₐ, and from the movements of the proximal section 3p of the catheter 3 between two determining times t_{d}, which movements are output by the detecting module 17.

To do so, the module 19 is able to deduce, from the movement of the proximal section 3p of the catheter 3 between two successive determining times t_{d}(k-1) and t_{d}(k) and from the map of the vascular system of the subject 5, the movement of the distal section 3d of the catheter 3 in the vascular system of the subject 5 between the two determining times t_{d}(k-1) and t_{d}(k).

The map of the vascular system of the subject 5 is for example determined beforehand by the module 19 from the initial image of the vascular system of the subject 5, which image is acquired by the imaging module 15.

Preferably, the determined position of the distal section 3d at each acquiring time tₐ is the position of this distal section 3d acquired by the imaging module 15. Moreover, at each determining time t_{d}(k) different from an acquiring time tₐ, the position of the distal section 3d is determined from the position of this distal section 3d at the immediately preceding determining time t_{d}(k-1) and from an estimation of the movement of the distal section 3d between the times t_{d}(k-1) and t_{d}(k).

Thus, the module 19 is able to determine the successive positions of the distal section 3d of the catheter 3 at the determining times t_{d} from the movements of the proximal section 3p, which movements are output by the detecting module 17, and to reset the position of this distal section 3d at each acquiring time tₐ, on the basis of the position acquired by the imaging module 15. This periodic resetting allows errors in the precision of the position such as determined by the detecting module 17 alone to be corrected.

The display means 11 comprise a displaying device 68 able to receive from the module 19 the successive positions of the distal section 3d of the catheter 3 at the determining times t_{d}, and to display, to a practitioner, at each determining time t_{d}, an image representative of the vascular system of the subject 5 and of the position of the catheter 3, and in particular its distal section 3d, with respect to this vascular system at this determining time t_{d}. An example of such an image is illustrated in FIG. 4. This image comprises a representation of the vascular system of the subject 5, on which representation there is superposed a representation of the distal section 3d of the catheter 3.

As illustrated in FIG. 2, in one embodiment, the processing and controlling unit 27, the processing unit 41 and the module 19 for determining the position of the distal section 3d are applications executed by a computer 72.

To this end, the computer 72 comprises a processor 78, one or more memories 80, human-machine interfacing means 82 and interfacing means 84.

The memory 80 comprises various memory regions containing applications intended to be executed by the processor 78, in particular applications corresponding to the functions executed by the processing and controlling unit 27 and/or the processing unit 41 and/or the module 19. The memory 80 also contains data relating to the vascular system of the subject 5, especially the initial image of the vascular system of the subject 5, which image is acquired by the imaging module 15, and the map of this vascular system, which map is determined by the module 19 from this initial image.

The processor 78 is suitable for executing applications contained in the memory 80 and especially an operating system allowing the conventional operational an information-technology system.

The computer 72 is able to exchange data with the emitter 23 and the detector 25 of the imaging module 15 and with the detector 40 of the detecting module 17 via the interfacing means 84. In particular, the interfacing means 84 comprise a wireless emitter/receiver able to exchange data with the communication interface 60 of the housing 50.

The human-machine interfacing means 82 comprise means 84 allowing an operator to input information for parameterizing the system 1 and the displaying device 68. In particular, the interfacing means 82 allow the user to define the frequency fₐ with which the position of the distal section 3d of the catheter is acquired by the imaging module 15.

An exemplary implementation of a method according to the invention by means of the system 1 for tracking the distal section of the catheter 3 during an intervention will now be described with reference to FIG. 5.

This method comprises an initial step 100 in which an initial image of the vascular system of the subject 5 is acquired by the imaging module 15 after a contrast agent that is opaque to x-rays has been introduced into the vascular system of the subject 5.

Moreover, in this initial step 100, the initial image is transmitted to the module 19, which determines, from this initial image, a map of the vascular system of the subject 5.

The initial image and the map of the vascular system are then stored in the memory 80 of the computer 72.

The intervention is then initiated, for example by the practitioner, in a step 102, by introducing the trocar 58 into a vein or artery of the vascular system through the skin of the subject 5, and by attaching this trocar 58 to the skin of the subject 5. The housing 50 is then fastened by its exterior connector 56 to the trocar 58, and the distal section 3d of the catheter 3 is introduced, through the duct 52 of the housing 50 and through the trocar 58, into the vascular system of the subject 5.

The distal section 3d of the catheter 3 is then moved in the vascular system of the subject 5, for example by an operator who moves the proximal section 3p of the catheter 3, in particular by translating this proximal section 3p toward the body of the subject 5 and/or by rotating this proximal section 3p about the longitudinal direction of the catheter 3.

The movement of the distal section 3d of the catheter 3 in the vascular system is then tracked by the system 1 and displayed, to the practitioner, by way of the following steps, which are carried out iteratively.

In an acquiring step 106, implemented at an acquiring time tₐ(n), the imaging module 15 acquires the position of the distal section 3d of the catheter 3 in the vascular system of the subject 5.

To do this, in a phase 108, the x-ray emitter 23 emits, at the acquiring time tₐ(n), x-rays in the direction of the region of interest of the body of the subject 5, in which region the catheter 3 is moved, in response to a command signal from the processing and controlling unit 27.

These rays pass through the body of the subject 5 and are then received by the detector 25. The detector 25 then sends electrical signals representative of the detected x-rays to the processing and controlling unit 27.

The unit 27 generates from these signals an x-ray image of the body of the subject 5, in which the distal section 3d of the catheter 3 appears.

The processing and controlling unit 27 then superposes the x-ray image thus generated on the initial image of the vascular system of the subject 5 in order to form an image in which both the vascular system and the catheter 3 appear.

Moreover, the unit 27 determines, from this reconstructed image, the position of the catheter 3, and in particular its distal section 3d, in the frame of reference R of the vascular system of the subject 5 at the acquiring time tₐ, and transmits this position to the module 19.

In a displaying phase 110, the module 19 transmits this position to the displaying means 11, which then display, to the practitioner, an image showing both the vascular system of the subject 5 and the distal portion 3d of the catheter 3 in this vascular system.

This acquiring step 106 is then repeated at the following acquiring time tₐ(n+1).

At each determining time t_{d}(k) comprised between these acquiring times tₐ(n) and tₐ(n+1), the position of the distal section 3d of the catheter 3 is determined, in a plurality of detecting steps 120, 121, from the movement of the proximal section 3p of this catheter 3, which movement is detected by the detecting module 17.

The detecting step 121 is thus reiterated to determine the position of the distal section 3d at each determining time t_{d}(k).

The detecting step 121 comprises a detecting phase 122 in which the module 17 detects movements of the proximal section 3p of the catheter 3, with respect to the vascular system of the subject 5, between the determining times t_{d}(k-1) and t_{d}(k). In the first iteration of the step 120, t_{d}(k-1) corresponds to the acquiring time tₐ(n).

In the phase 122, the movement detector 40 determines the rotational movements of the proximal section 3p of the catheter about its longitudinal direction and the translational movements of the catheter of the proximal section 3p in its longitudinal direction, with respect to this detector 40, between the two times t_{d}(k-1) and t_{d}(k).

To do this, the laser emitter 42 emits a laser beam toward the detecting region 43, through which the catheter 3 runs. The laser beam, reflected by the exterior wall of the catheter 3, is received by the optical receiver 44.

The optical receiver 44 thus acquires at multiple receiving times tᵣ between the determining times t_{d}(k-1) and t_{d}(k) images of the section of the catheter 3 passing through the region 43, and transmits this information to the processing unit 41 by the wireless communication interface 60.

The processing unit 41 analyzes these images to determine the relative translational and rotational movement of the proximal section 3p of the catheter 3 with respect to the detector 40 between the determining times t_{d}(k-1) and t_{d}(k), and deduces therefrom the relative movement of the proximal section 3p of the catheter 3 in the frame of reference R of the vascular system of the subject 5. The processing unit 41 transmits this information to the module 19.

The detecting phase 122 is followed by a phase 124 in which the module 19 determines the position of the distal section 3d of the catheter 3, at the determining time t_{d}(k), from the position of this distal section 3d at the determining time t_{d}(k-1) and from the movement of the proximal section 3p of the catheter 3 between the determining times t_{d}(k) and t_{d}(k-1).

To do this, the module 19 determines, from the movement of the proximal section 3p of the catheter 3 between the times t_{d}(k) and t_{d}(k-1) and from the map of the vascular system stored in the memory 80, the movement of the distal section 3d of the catheter 3 in the vascular system of the subject 5 between the times t_{d}(k) and t_{d}(k-1).

The module 19 then determines the position of the distal section 3d at the time t_{d}(k) from the position of this distal section at the time t_{d}(k-1) and from an estimation of the movement of the distal section 3d between the times t_{d}(k-1) and t_{d}(k).

In a displaying phase 126, the module 19 transmits this position to the display means 11, which then display, to the practitioner, an image showing both the vascular system of the subject 5 and the catheter 3 in this vascular system.

The system and method according to the invention thus allow images to be displayed to the practitioner, these images illustrating the movement of the catheter that the practitioner is manipulating in the vascular system of the subject 5 at a satisfactory frequency, while decreasing the frequency of emission of x-rays toward the body of the subject 5 and therefore decreasing the risks to which the subject 5 is exposed. The system according to the invention moreover has the advantage of being of low cost.

Furthermore, the housing 50 is miniaturized, thereby making it easier to manipulate, in particular during an intervention.

It will be understood that the exemplary embodiments presented above are nonlimiting.

In particular, the system according to the invention may be used to track the movement of a plurality of medical instruments in the body of the subject, for example to track the movement of a catheter and a micro-catheter, the micro-catheter being inserted and moved inside the catheter.

The system 1 then includes a plurality of detectors 40, each able to determine the relative movement of an associated medical instrument with respect to another medical instrument or with respect to the body of the subject.

Each detector 40 is contained in a housing 50 that is either held stationary or movable with respect to the body of the subject.

The movement of each medical instrument with respect to the body of the subject is then determined by composing the movement of this medical instrument with respect to the associated housing 50, which movement is determined by the detector 40 contained in this housing, and the movement of the associated housing 50.

For example, to track the movement of a catheter and a micro-catheter inserted and moved inside the catheter, the system comprises a first housing associated with the catheter and held stationary with respect to the body of the subject, and a second housing associated with a micro-catheter and held stationary with respect to the catheter.

The first housing allows the movement of the catheter with respect to the body of the subject to be determined.

The second housing allows the movement of the micro-catheter with respect to the second housing, and therefore the movement of this micro-catheter with respect to the catheter, to be determined. The movement of the catheter with respect to the body of the subject is then determined by composing the movement of the micro-catheter with respect to the catheter and of the movement of the catheter with respect to the body of the subject.

Furthermore, according to one variant, the detector 40 and the processing unit are connected by a wired link, and the data captured by the detector 40 are transmitted to the processing unit 41 via this wired link.

Of course, other embodiments may be envisioned, and the technical features of the aforementioned embodiments and variants may be combined together.

1. A system for tracking a first section of a medical instrument, which section is inserted into the body of a subject, while it is being moved in the body of the subject, said system comprising:
means for determining a position of said first section with respect to the body of the subject at at least one determining time (t_{d}); and
means for displaying, to a user, at said determining time (t_{d}), an image representative of at least one part of the body of the subject and of the first section of the medical instrument in the position of the first section determined by said determining means at the determining time (t_{d}), the system being characterized in that said determining means comprise:
an imaging module able to acquire, at at least one acquiring time (tₐ) prior to said determining time (t_{d}), a position of the first section of the medical instrument with respect to the body of the subject;
a module for detecting a movement of a second section of the medical instrument with respect to the body of the subject between said acquiring time (tₐ) and said determining time (t_{d}); and
a determining module able to determine, from the position of the first section at said acquiring time (tₐ), which position is output by said imaging module, and from said movement of the second section of the medical instrument between said acquiring time (tₐ) and said determining time (t_{d}), which movement is detected by said detecting module, the position of the first section of the medical instrument with respect to the body of the subject at said determining time (t_{d}), said determining module being able to determine the position of the first section of the medical instrument with respect to the body of the subject, at each of a plurality of successive determining times (t_{d}) comprised between first and second successive acquiring times (tₐ(n−1)), (tₐ(n)), from the position of the first section at said first acquiring time (tₐ(n−1)), which position is output by said imaging module, and from the movement of the second section of the medical instrument between said first acquiring time (tₐ(n−1)) and each determining time (t_{d}) of said plurality of determining times (t_{d}), which movement is detected by said detecting module.

2. The tracking system as claimed in claim 1, characterized in that said detecting module is able to detect a translation of said second section of the medical instrument in its longitudinal direction and a rotation of said second section of the medical instrument about its longitudinal direction with respect to the body of the subject between said acquiring time (tₐ) and said determining time (t_{d}).

3. The tracking system as claimed in claim 1, characterized in that said detecting module comprises at least one detector of a movement of the second section with respect to this detector.

4. The tracking system as claimed in claim 3, characterized in that said detector is contained in a housing including a duct for passing the medical instrument.

5. The tracking system as claimed in claim 4, characterized in that said housing includes a first section enclosing said detector and a second section enclosing said passing duct.

6. The tracking system as claimed in claim 5, characterized in that said second section is leaktight, said medical instrument passing through said passing duct being sealably isolated from said first section.

7. The tracking system as claimed in claim 6, characterized in that said second section is removably mounted on said first section.

8. The tracking system as claimed in claim 3, characterized in that said detector is an optical detector.

9. The tracking system as claimed in claim 8, characterized in that said optical detector comprises at least one light source able to emit an incident light beam onto a region of the second section of the medical instrument and one optical receiver able to detect a light beam reflected by the second section of the medical instrument.

10. The tracking system as claimed in claim 9, characterized in that said light source is able to emit the incident light beam onto a region of the second section of the medical instrument during the passage of said second section through said passing duct.

11. The tracking system as claimed in claim 3, characterized in that said detector is movable with respect to the body of the subject, and in that said detecting module comprises means for detecting a movement of the detector with respect to the body of the subject.

12. The tracking system as claimed in claim 1, characterized in that said first section of the medical instrument comprises at least one region visible by optical imaging, and in that said imaging module comprises an emitter able to emit optical rays toward the body of the subject, and a detector able to receive the optical rays emitted by said emitter through the body of the subject.

13. The tracking system as claimed in claim 1, characterized in that said detecting module is able to detect a movement of the second section of the medical instrument with respect to the body of the subject, between said acquiring time (tₐ) and said determining time (t_{d}), said second section being outside the body of the subject.

14. A method for tracking a first section of a medical instrument inserted into the body of a subject as it is moved in the body of the subject, comprising:
determining a position of said first section with respect to the body of the subject at at least one determining time (t_{d}); and
displaying, to a user, at each determining time (t_{d}), an image representative of at least one part of the body of the subject and of the first section of the medical instrument in the position of the first section (determined by said determining means at the determining time (t_{d}),
the method being characterized in that the determination of the position of said first section comprises:
acquiring, at at least one acquiring time (tₐ) prior to said determining time (t_{d}), a position of the first section of the medical instrument with respect to the body of the subject;
detecting a movement of a second section of the medical instrument with respect to the body of the subject between said acquiring time (tₐ) and said determining time (t_{d}); and
determining, at each of a plurality of successive determining times (t_{d}) comprised between first and second successive acquiring times (tₐ(n−1)), (tₐ(n)), the position of the first section of the medical instrument with respect to the body of the subject, from the position of the first section at said first acquiring time (tₐ(n−1)) and from the movement of the second section of the medical instrument between said first acquiring time (tₐ(n−1)) and each determining time (t_{d}) of said plurality of determining times (t_{d}).
